(19) United States
(12) Patent Application Publication
(10) Pub. No.: US 2016/0256218 A1
(43) Pub. Date: Sep. 8, 2016

(54) Perivascular Electroporation Device and Method for Extending Vascular Patency

(71) Applicant: THE BOARD OF TRUSTEES OF THE LELAND STANFORD JUNIOR UNIVERSITY, Palo Alto, CA (US)

(72) Inventors: Viral L. Gandhi, Costa Mesa, CA (US); Christian B. Moyer, Palo Alto, CA (US); Andrew L. Mesher, Seattle, WA (US); Elisabeth K. Wynne, St. Louis, MO (US)

(21) Appl. No.: 15/060,176

(22) Filed: Mar. 3, 2016

Related U.S. Application Data
(60) Provisional application No. 62/128,379, filed on Mar. 4, 2015.

(51) Int. Cl. A61B 18/18; A61B 18/12

(52) U.S. Cl. CPC A61B 18/18; A61B 18/1206; A61B 2018/00577

(57) ABSTRACT

A system for performing perivascular electroporation of a blood vessel may include a tissue treatment device configured to contact and surround at least part of a circumference of an outer surface of a blood vessel wall and a control device coupled with the tissue treatment device. The control device may include an electric pulse generator and a tissue impedance modulator. A method for performing perivascular electroporation of a blood vessel may involve coupling a tissue treatment device of a perivascular electroporation system with an outer surface of a wall of the blood vessel and delivering electric pulses to an outermost layer of the blood vessel wall, while limiting a depth of penetration of the electric pulses such that they do not reach an innermost layer of the blood vessel wall.

### CROSS-REFERENCE TO RELATED APPLICATION

This application claims priority to U.S. Provisional Patent Application No. 62/128,379, filed Mar. 4, 2015, and entitled “Perivascular Electroporation Device and Method for Extending Vascular Patency.” The entirety of U.S. Provisional Patent Application No. 62/128,379 is herein incorporated by reference.

### FIELD OF THE INVENTION

The present invention comprises a device and method to extend vascular patency using perivascular electroporation. More particularly, the present invention relates to a set of electrodes for electroporating the outer wall of a vessel to extend said vessel's patency.

### BACKGROUND

Blood vessels may experience diminished patency as a result of naturally occurring processes or from the body's response to introduced materials or devices. In many instances, diminished patency results at least in part from vascular cell proliferation in response to an injury caused by an intervention or open surgery involving vascular structures. Areas where two blood vessels come together (“anastomotic junctions”) and areas near anastomotic junctions are at an especially significant risk of occlusion, due to vascular cell proliferation, generally referred to as neointimal hyperplasia.

Anastomotic junctions exist, for example, in vascular fistulas and grafts, which are used in a wide variety of circumstances to re-configure or re-establish vascular circulation in a patient. For example, fistulas and grafts are used to create access sites for blood withdrawal and return in patients undergoing periodic kidney dialysis, hemofiltration, and other extracorporeal blood treatments. Usually, either a native artery and vein are connected together via a side-to-side anastomosis, or a saphenous vein or synthetic graft is placed between an artery and a vein and attached at each end via an end-to-side anastomosis. Natural grafts (usually a vein harvested from the patient being treated) and synthetic grafts are also used in a number of open and minimally invasive surgical procedures for treating vascular disorders, such as coronary artery bypass grafting for treating heart disease, surgical graft introduction for treating abdominal aortic aneurysms, peripheral vasculature repair, and the like. In all cases, at least two anastomotic connections are required for implanting the graft. Neointimal hyperplasia will often occur as a response to the elevated hemodynamics in and around the anastomosis, causing patency issues for nearly 50% of patients undergoing these procedures at one year.

At present, there are no effective treatments for hyperplasia near anastomotic junctions in any of the cases discussed above. When an arterio-venous (A-V) fistula or graft fails in a dialysis patient, it is necessary to create a new dialysis access site. After multiple A-V fistula sites have been tried on a patient and no additional sites are available, kidney dialysis is simply no longer available for that patient. While it is possible for heart bypass patients having failed grafts to redo the procedure, second and later procedures are seldom as effective in treating the disease as the initial bypass procedure. Moreover, the availability of autologous blood vessels for performing the procedure limits the number of procedures that can be performed.

Unfortunately, no one method or approach appears to adequately address the challenges of vascular patency management. Accordingly, the need remains to identify an approach that enables mitigation of the host response to vascular procedures and/or implanted devices and thereby maintains patency of the vasculature at or near the site of such activities. Towards this end, continuous electric fields have been noted to affect the migration of certain vascular cell types in vitro, e.g. Bai, et al. (Arterioscler Thromb Vasc Biol, Vol 24, pp 1234-39, 2004). Using a different approach, Burwell et al. (U.S. Pat. No. 7,730,894) teach that photonic irradiation may be employed to advantageously affect vascular tissue in photodynamic therapy. However, the method taught is not applicable for extended use in vivo and requires additional agents. Conventional thermal, chemical, and other ablation techniques have been employed for the treatment of a variety of undesirable tissue. High temperature thermal therapies have the advantage of ease of application. However, the disadvantage is that the extent of the treated area is difficult to control, because blood circulation has a strong local effect on the temperature field that develops in the tissue. Also, many of the current techniques are designed only for ablating an artery and not necessarily an artery/vein link.

Therefore, it would be very desirable to have methods and systems for preventing stenosis near anastomotic junctions, such as those formed as part of an arterio-venous fistula, bypass graft or other graft in a patient's vasculature. It would be particularly desirable to provide methods and systems suitable for treating arterio-venous connections at the time they are created, to effectively inhibit hyperplasia prior to the start of the host response cascade. Preferably, the methods and systems for inhibiting hyperplasia would require little or no modification to the implantation techniques themselves and would be suitable for use in a wide variety of procedures that rely on the formation of arterio-venous attachments, including those described above. At least some of these objectives will be met by the embodiments described hereinafter.

### BRIEF SUMMARY

The present application describes a method and system for decellularizing a blood vessel near an anastomosis, using a highly-specific, minimally invasive, surgical technique called perivascular electroporation. Electroporation is a technique used to make cell membranes permeable by exposing them to electric pulses. “Perivascular” refers to the placement of an electrical pulse generating device on the exterior of the blood vessel (perivascular). The application of electrical pulses causes permeabilization of cells making up a portion of the blood vessel, preferentially in the outer layers of the vessel and less preferentially in the inner layers of the vessel. The electrical pulses irreversibly permeate the vascular cell membranes, thereby invoking cell death through an apoptotic (non-necrotic) signaling pathway. The length of time for transmitting the electrical pulses, the voltage applied, and the resulting membrane permeability are all controlled within defined ranges. The irreversibly permeabilized cells may be left in situ and may be removed by natural processes, such as the body's own immune system. The amount of vascular decellularization achievable through the use of perivsacular electroporation in a portion of a blood vessel, without inducing thermal damage, may be considerable.

Perivascular electroporation in blood vessels to decellularize a portion of the vessel is different from other forms of electrical therapies and treatments. An electrical pulse can either have no effect on the cell membrane, effect internal cell components, reversibly open the cell membrane, after which the cells can survive, or irreversibly open the cell membrane, after which the cells die. Perivascular electroporation is different from intracellular electro-manipulation, which substantially only affects the interior of the cell and does not cause cell membrane damage. Perivascular electroporation is not electrically induced thermal coagulation, which induces cell damage through thermal effects, but rather a more benign method to disrupt only the cell membrane of cells in a targeted region of a vessel wall. Perivascular electroporation that irreversibly disrupts the cell membrane is also different from electrochemotherapy, in which reversible electroporation pulses are used to introduce drugs into living cells.

Perivascular electroporation uses electrical pulses to create vascular decellularization by disrupting or permeabilizing the cell membrane in the outer portions of a target vessel. Perivascular electroporation is different from perivascular ablation, which aims to destroy cells through thermal effects and create instantaneous necrosis. Perivascular ablation techniques are described, for example, in U.S. Pat. No. 8,048,067 and U.S. Patent Application Pub. No 2012/0109023. In cases of perivascular ablation, the necrotic vessel stiffens and impairs future dilation under high-pressure hemodynamic states. Perivascular electroporation avoids tissue necrosis by opening the cellular membrane without lysing the cell, inducing cells to undergo an apoptotic rather than necrotic signaling pathway. The decellularized vessel retains the extracellular structure and compliance of the native vessel.

To achieve electroporation of blood vessel cells, an electrical pulse may be delivered to a vessel via the vessel lumen (endovascular electroporation) or the exterior of the vessel (perivascular electroporation). Of these delivery paths, endovascular approaches have been generally preferred over perivascular approaches, because they could be performed using catheters passed through the blood vessels and thus avoid open surgical procedures. Endovascular approaches are described, for example, in U.S. Patent Application Pub. Nos. 2001/0044596, 2009/0247933 and 2010/0004623. These references describe endovascular electroporation techniques that apply a therapy originating from the vessel lumen and traveling transmurally to the outer layer of the vessel. Thus, the methods described in these references damage the endothelial layer as part of a transmural electroporation therapy. One of the challenges with methods that damage the endothelium is that this damage elicits a host immune response and increases the risk of thrombosis following an arterio-venous connection. Perivascular electroporation mitigates this risk by decellularizing the vessel preferentially in the outer layers of the vessel and preserving cells in the inner layers of the vessel, specifically the endothelial layer (intima).

The embodiments described herein relate to a method and system for use on an outer surface of a blood vessel—in other words, a perivascular approach. The method and system may often be applied to an exposed vessel, such as one exposed during an open surgical procedure. Such surgical procedures include, but are not limited to, arteriovenous fistula creation, arteriovenous graft creation, peripheral vascular bypass, and coronary artery bypass grafting.

A number of prior art methods seek to mitigate the host response to arterio-venous anastomoses by administering a therapy over an extended time period, for example with an implantable drug or device. For example, several implantable devices have been developed to mitigate host response by altering anastomosis shape (e.g., U.S. Pat. Nos. 8,366,651 and 8,690,816 and U.S. Patent Application Pub. Nos. 2013/0197546 and 2014/0180191), modulating hemodynamics (e.g., U.S. Pat. Nos. 7,025,741, 8,114,044 and 8,764,698), or releasing an anti-proliferative agent over time (e.g., U.S. Pat. No. 7,807,191 and U.S. Patent Application Pub. No. 2014/0249618). Implantable devices, however, expose the blood vessel to high risk of infection and thrombosis. Perivascular electroporation, in contrast, is a one-time therapy performed at the time of arterio-venous anastomosis creation. Its effects are long lasting, and it does not require an implant, thus decreasing the risk of infection and thrombosis.

### BRIEF DESCRIPTION OF THE DRAWINGS

FIG. 1 is a perspective, schematic view of a perivascular electroporation system for a blood vessel, according to one embodiment;

FIG. 2 is a flow chart of a perivascular electroporation method for a blood vessel, according to one embodiment;

FIG. 3 is a schematic diagram of the perivascular electroporation system of FIG. 1;

FIG. 4A is an end-on, schematic view of a blood vessel, indicating the various layers of the blood vessel wall;

FIG. 4B is an end-on, schematic view of the blood vessel of FIG. 4A, with multiple electrodes and impedance modulators disposed around its circumference, according to one embodiment;

FIG. 4C is an end-on, schematic view of a portion of the blood vessel of FIG. 4B, illustrating electrical pathways emanating from the electrodes, according to one embodiment; and

FIGS. 5A-5C are perspective views of a tissue treatment portion of a perivascular electroporation system, according to one embodiment.

### DETAILED DESCRIPTION OF THE INVENTION

The following description of various embodiments should not be used to limit the scope of the invention as defined by the claims. The embodiment descriptions are provided for exemplary purposes only. Alternative embodiments, which may or may not be described below, may include different features or combinations of features, without departing from the scope of the invention.

As discussed above, this disclosure describes various embodiments of a method and system for treating a blood vessel with perivascular electroporation, from the outside of the cell in, towards the cell lumen, in order to cause cell death, without harm to the blood vessel extracellular matrix, in order to prevent neointimal hyperplasia and reduce vascular stenosis and restenosis at the site of treatment. In various embodiments, any blood vessel or type of blood vessel—artery, vein, graft, fistula, etc.—may be treated, using the systems and methods described herein.

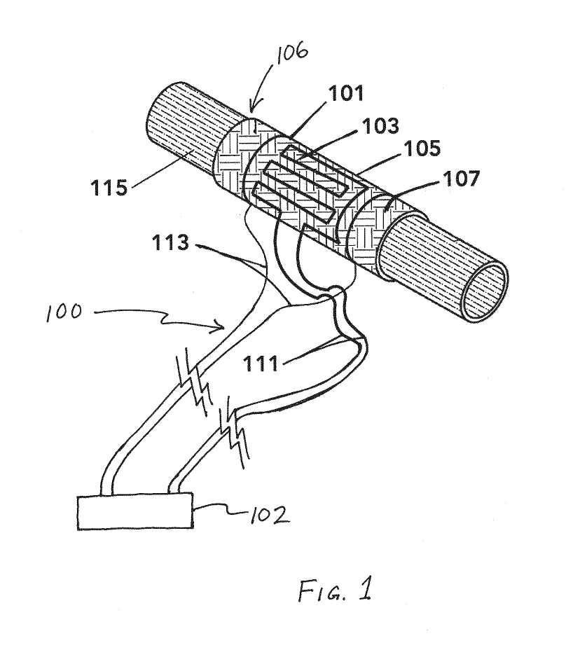

Referring to FIG. 1, one embodiment of a perivascular electroporation system 100 is illustrated schematically, attached to a portion of a blood vessel 106. The blood vessel 106 is shown in partial cross section, so that the tunica adventitia 107 (or “outermost layer”) and the tunica media 115 of the blood vessel 106 are visible. The system 100 may include a tissue treatment portion 101, a controller 102 (or “box”) and one or more connectors 111, 113 connecting the tissue treatment portion 101 with the controller 102. The tissue treatment portion 101 may include a substrate 105 (or “housing”), which may contain multiple electrodes, for example in an electrode array (not visible in FIG. 1), for delivering the electrical energy used in the electroporation procedure and impedance modulation electronics 103, for modulating impedance during electroporation. The electrodes may be connected to the controller 102 via a first set of wires 113, and the impedance modulation electronics 103 may be connected to the controller 102 via a second set of wires 111. Any suitable number and type of wires may be used.

The embodiment in FIG. 1 includes one controller 102, but alternative embodiments may include separate controllers, for example one for electroporation therapy delivery and one for impedance modulation. The controller 102 in FIG. 1 is not drawn to scale, and in fact, any of the drawing figures may include features that are not drawn to scale. Generally, the controller 102 includes a pulse generator and an impedance modulator, both of which are used to deliver treatment via the tissue treatment portion 101. The controller 102 may be pre-programmed to provide a set, predetermined pulse therapy. Alternatively, the controller 102 may in some embodiments be adjustable by a user.

The tissue treatment portion 101 may be designed to wrap completely or partially around the outer surface of the tunica adventitia 107 of the blood vessel 106. As such, the substrate 105 of the tissue treatment portion 101, as well as any or all of the components attached to or housed within the substrate 105, may be made of a material that makes it easy to wrap the tissue treatment portion 101 around the blood vessel 106. For example, in some embodiments, the substrate 105 may be made of a shape memory material that may be stretched into an approximately flat shape for passing under or past the vessel, and that may then be released from constraint to assume its default shape and thus wrap around the vessel. In general, the tissue treatment portion 101 may have any suitable shape, size or configuration that might lend itself for contacting and at least partially surrounding a blood vessel 106.

Once the tissue treatment portion 101 is positioned around the blood vessel 106, the perivascular electroporation system 100 may be used to deliver an electroporation pulse sequence generated by a pulse generator in the controller 102. The pulse sequence will typically be preset in the controller 102. However, in alternative embodiments, the pulse sequence may be adjustable by a user, such as a physician. The pulse sequence electroporation will result in target cell permeabilization, starting in the tunica adventitia 107 and extending to the tunica media 115. Cell permeabilization may be modulated by the impedance modulation electronics 103, which are connected to the pulse generator via wires 111, and which are controlled by the controller 102. The system 100 may use impedance modulation to modulate the impedance of the blood vessel wall tissue, in order to protect the tunica intima (the innermost layer) of the blood vessel wall.

In general, the system 100 may be used to direct electroporation therapy from the outside of the vessel wall inward, toward the vessel lumen, but without reaching the innermost layer of the vessel wall. Perivascular electroporation therapy delivered by the system 100 will typically result in eventual cell death of the tunica adventitia and tunica media, without causing coagulative necrosis and while maintaining the cellularity of the tunica intima the extracellular structure of the blood vessel.

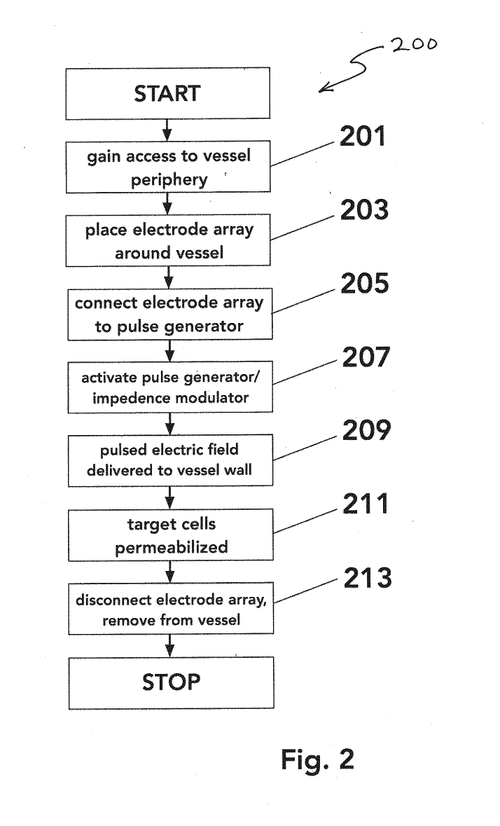

Referring now to FIG. 2, one embodiment of a method 200 for perivascular electroporation of a blood vessel is described. This embodiment involves perivascular electroporation during an open surgical procedure (e.g. arterio-venous fistula creation, arterio-venous grafting, coronary artery bypass grafting, peripheral arterial bypass grafting, etc), although in alternative embodiments, the method 200 or a variation thereon may be performed as part of a minimally invasive, less invasive or even transvascular procedure. In the embodiment of FIG. 2, the method 200 begins by gaining access to the outside/peripheral wall of a blood vessel 201, during an open surgical procedure. In some cases, the blood vessel wall will be dissected free of surrounding tissues and thus can be accessed circumferentially for a predetermined length. Once the blood vessel is accessed, an electrode array (or more generally the tissue treatment portion 101) of the treatment device may be placed around the blood vessel 203, often in a predetermined orientation and configuration. The orientation will be indicated by the delivery system, and the configuration of the tissue treatment portion 101 may include, but is not limited to, a sleeve, a malleable sheet, an extended J-shape, two or more opposing rigid structures, the inner layer of a tube shaped inflatable structure, a single contiguous malleable filament, multiple malleable filaments, or an outer cylinder with internally radially directed filaments.

Next, in some embodiments, tissue treatment portion 101 may be connected to the impedance modulation pulse generator 205 (or the controller 102). In alternative embodiments, however, the tissue treatment portion 101 may already be attached to the controller 102. At this point, the user/operator may activate the pulse generator/impedance modulator 207 (i.e., the controller 102) to start a treatment. In various embodiments, the system 100 delivers a predetermined pulse sequence electric field 209 to the vessel wall, with or without impedance modulation, depending on the specific instance of therapy. After delivery of the pulsed electric field 209, the target cells of the blood vessel will be permeabilized 211, eventually resulting in cell death. After completion of the pulsed electric field, the tissue treatment portion 101 of the system 100 may be removed from the outside of the blood vessel wall 213 atraumatically, leaving the structure of the blood vessel completely intact.

Referring now to FIG. 3, a schematic diagram of the perivascular electroporation system 100 described above in relation to FIG. 1 is presented. In this embodiment, the controller 102 of the system 100 includes a power supply 302, a pulse output circuit 304 (or “pulse generator”), and a tissue impedance modulator 306. The tissue treatment portion 101 includes an electrode array 308 and an impedance modulator delivery device 310, both of which are used together to deliver the electroporation electric energy to the blood vessel outer wall and control delivery of the energy. The pulse output circuit 304 may incorporate multiple parameters of electric field pulse generation, including but not limited to a pulse timer 312, pulse sequence cycles 314, and output amplitude 316. These parameters 312, 314, 316 allow for refinement and control of the signal to the electrodes that deliver the pulsed electric fields to the target tissue. In some embodiments, the pulse timer 312 may have a range of about 0.5 Hz to about 10 Hz, the pulse sequence cycles 314 may number from about 1 to about 100, and the output amplitude may range from about 1 V/cm to about 10,000 V/cm. These parameters 312, 314, 316 are only provided as examples, and any other suitable parameters or combinations of parameters may be used.

The tissue impedance modulator 306 may receive input in the form of tissue parameters 305, such as but not limited to tissue depth, temperature, consistency, electrolyte levels, pH levels, and/or any other suitable tissue parameters that can be obtained previous to and/or during the perivascular electroporation procedure. The output of the tissue impedance modulator 306 is a signal that activates the impedance modulator delivery device 310. This output may include, but is not limited to, electric fields, temperature regulation, pH regulation, and/or liquid or gaseous substance application to the site of therapy. In various alternative embodiments, the controller 102 and the tissue treatment portion 101 may be coupled to one another permanently or may be detachable from one another.

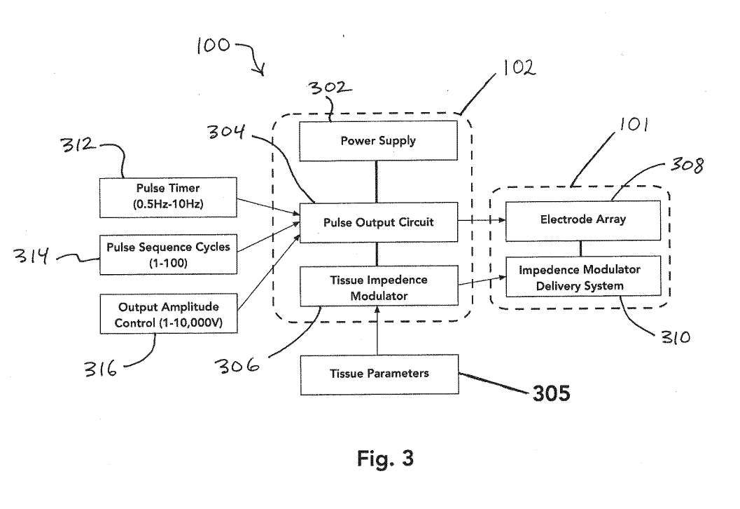

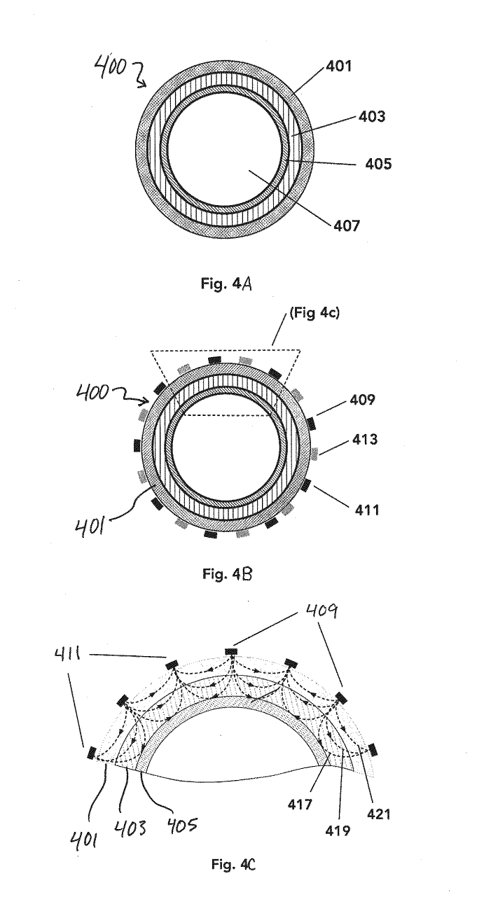

FIG. 4A is an end-on, schematic representation of a blood vessel 400, illustrating the various layers of the vessel wall. As described previously, the layers of the blood vessel wall generally include the tunica adventitia 401 (outermost layer), the tunica media 403 (middle layer) and the tunica intima 405 (inner layer). The interior of the blood vessel 400 is referred to as the lumen 407, where liquid substances such as blood flow. Potential target cell types of the blood vessel wall for the perivascular electroporation method described herein include, but are not limited to, fibroblasts, smooth muscle cells, myofibroblasts, mesenchymal stem cells, and other neointimal progenitor cells.

FIG. 4B is the same end-on, schematic representation of the blood vessel 400, but also shows components of a tissue treatment device applied circumferentially around the outer surface of the tunica adventitia 401. In this embodiment, the tissue treatment device includes an electrode array with longitudinally disposed electrodes. The electrode array includes positive nodes 409 and negative nodes 411. The tissue treatment device also includes longitudinally disposed impedance modulation electronics 413, so the impedance modulation portion of the system and the electrode array delivering the pulsed electric field, which results in cell permeabilization of targeted tissues, are potentially but not exclusively interconnected.

FIG. 4C is a magnified view of the circled portion of the blood vessel wall in FIG. 4B. FIG. 4C shows electric field lines 417, 419, 421 passing from positive nodes 409 to negative nodes 411 of the electrode array. The impedance modulation delivery device 413 acts to guide the electric fields, so that the tunica adventitia 401 and the tunica media 403 are treated, while the tunica intima 405 is protected from the electric fields during permeabilization. In other words, all the electric fields 419, 421, 423 are contained within the tunica adventitia 401 and tunica media 403, to result in the permeabilization of cells starting from the tunica adventitia 401 and proceeding into the tunica media 403, without affecting the tunica intima 405.

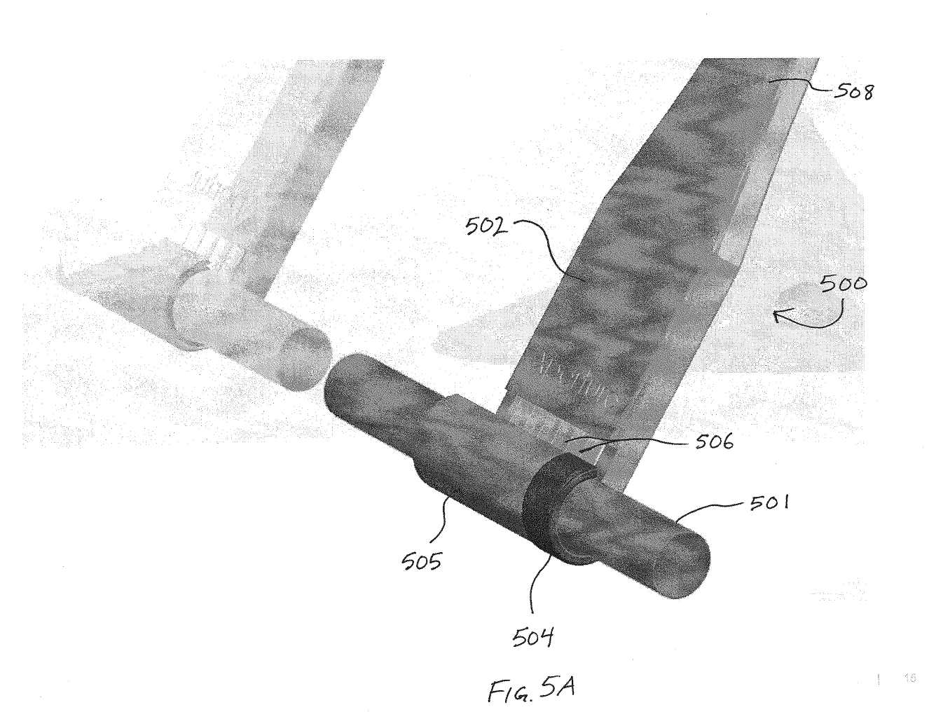

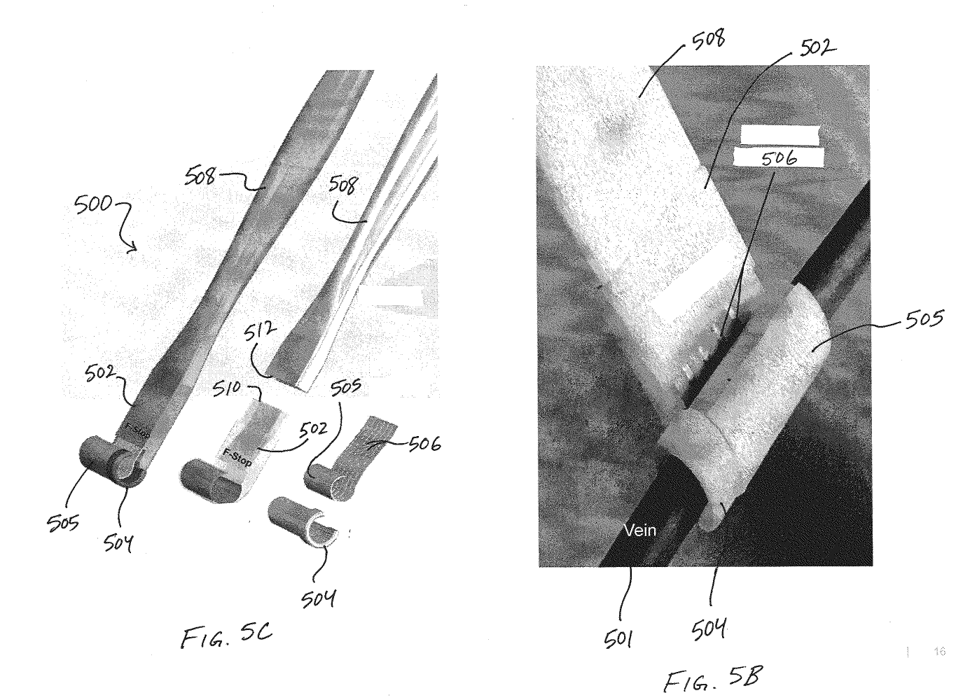

Referring now to FIGS. 5A-5C, another embodiment of a tissue treatment device 500 of a perivascular electroporation system is illustrated. The tissue treatment device 500 may also be referred to as a probe, a tissue contact device, an energy delivery device, or any other suitable terminology. In the illustrated embodiment, the tissue treatment device 500 includes a distal tissue contact portion 502 and a proximal shaft 508. Although not illustrated in FIGS. 5A-5C, the device 500 may also include a handle on the end of the shaft 508 that is opposite the tissue contact portion 502. Generally, the tissue contact portion 502 may have a flat configuration, for easy positioning around a blood vessel 501, and may also include a curved distal end for circling around the vessel 501. In some embodiments, the tissue contact portion may also include a rigid, semi-circular support member 504 and a flexible electrode pad 505, which holds multiple electrodes 506 disposed in an array. The flexible electrode pad 505 may fit around the support member 504. The electrodes 506 may be exposed on the inner surface of the tissue contact portion 502, so that they contact the blood vessel wall 501. As illustrated in FIG. 5C, in one embodiment, the tissue contact portion 502 may plug into the shaft 508 via a plug portion 510 on the tissue contact portion 502 and a receptacle 510 on the shaft 508. In other embodiments, the tissue contact portion 502 and the shaft 508 may be formed as a monolithic unit or may be permanently attached to one another. In the illustrated embodiment, the electrodes 506 are disposed in a circumferential pattern on the electrode pad 505 and thus on the tissue contact portion 502. In alternative embodiments, as mentioned above in relation to FIGS. 4B and 4C, electrodes 409, 411 may be disposed in a longitudinal array, rather than a circumferential array.

As with previously described embodiments, the embodiment of the tissue treatment device 500 illustrated in FIGS. 5A-5C is only one possible embodiment, and many variations are contemplated.

The above description is not intended to limit the meaning of the words used in the following claims that define the invention. Rather, it is contemplated that future modifications in structure, function or result will exist that are not substantial changes and that all such insubstantial changes in what is claimed are intended to be covered by the claims. Likewise, various changes, additions, omissions, and modifications can be made to the illustrated embodiments without departing from the spirit of the present invention.

What is claimed is:

1. A system for performing perivascular electroporation of a blood vessel, the system comprising:
a tissue treatment device configured to contact and surround at least part of a circumference of an outer surface of a blood vessel wall; and
a control device coupled with the tissue treatment device, the control device comprising:
an electric pulse generator configured to deliver an electric pulse to the tissue treatment device of between 1 V/cm and 10,000 V/cm; and
a tissue impedance modulator coupled with the electric pulse generator.

2. A system as in claim 1, wherein the control device further comprises a power supply coupled with at least the electric pulse generator.

3. A system as in claim 1, wherein the tissue treatment device comprises:
a substrate configured to contact and surround the blood vessel wall; and
an electrode array coupled with the substrate so as to contact the blood vessel wall when the substrate is positioned around the blood vessel wall.

4. A system as in claim 3, wherein the tissue treatment device further comprises impedance modulation electronics for modulating the electric pulses.

5. A system as in claim 3, wherein at least part of the substrate comprises a material selected from the group consisting of a flexible material, a malleable material and a shape memory material.

6. A system as in claim 3, wherein the substrate comprises a structure selected from the group consisting of a sleeve, a malleable sheet, an extended J-shape, two or more opposing rigid structures, the inner layer of a tube shaped inflatable structure, a single contiguous malleable filament, multiple malleable filaments, and an outer cylinder with internally radially directed filaments.

7. A system as in claim 3, wherein the electrode array is coupled with an electrode pad.

8. A system as in claim 1, wherein the tissue impedance modulator is configured to receive signals related to one or more tissue parameters of the blood vessel.

9. A system as in claim 8, wherein the one or more tissue parameters are selected from the group consisting of tissue depth, temperature, consistency, electrolyte level, and pH level.

10. A method for performing perivascular electroporation of a blood vessel, the method comprising:
coupling a tissue treatment device of a perivascular electroporation system with an outer surface of a wall of the blood vessel;
delivering electric pulses to an outermost layer of the blood vessel wall, using an electrode array on the tissue treatment device, while limiting a depth of penetration of the electric pulses such that they do not reach an innermost layer of the blood vessel wall,
wherein delivering the electric pulses causes cell permeabilization of at least a portion of cells comprising the outermost layer of the blood vessel wall.

11. A method as in claim 10, wherein coupling the tissue treatment device with the outer surface comprises wrapping the tissue treatment device around at least a portion of a circumference of the blood vessel.

12. A method as in claim 10, wherein limiting the depth comprises modulating tissue impedance of the blood vessel wall, using a tissue impedance modulator of the tissue treatment device.

13. A method as in claim 10, further comprising sending electric pulse signals and tissue impedance modulation signals from a controller to the tissue treatment device.

14. A method as in claim 13, wherein the tissue impedance modulation signals are selected from the group consisting of electric fields, temperature regulation, pH regulation, and liquid or gaseous substance application.

15. A method as in claim 13, further comprising receiving signals related to one or more tissue parameters with a tissue impedance modulator of the controller, wherein the one or more tissue parameters are selected from the group consisting of tissue depth, temperature, consistency, electrolyte level, and pH level.

16. A method as in claim 10, wherein delivering the electric pulses comprises delivering the pulses to the outermost layer and to a middle layer of the blood vessel wall but not to the innermost layer.

17. A method as in claim 10, wherein delivering the electric pulses comprises delivering pulses in a range of 1 V/cm to 10,000 V/cm.
